(19) United States
(12) Patent Application Publication
(10) Pub. No.: US 2013/0184783 A1
(43) Pub. Date: Jul. 18, 2013

(54) HYBRID BIOORGANIC INTERFACE FOR NEURONAL PHOTOACTIVATION, AND RETINAL PROSTHETIC DEVICE

(71) Applicants: Maria Rosa ANTOGNAZZA, Venegono Inferiore (Varese) (IT); Diego Ghezzi, Genova (IT); Marco Dal Maschio, Genova (IT); Erica Lanzarini, Santa Sofia (Forli-Cesena) (IT); Guglielmo Lanzani, Milano (IT); Fabio Benfenati, Genova (IT)

(72) Inventors: Maria Rosa ANTOGNAZZA, Venegono Inferiore (Varese) (IT); Diego Ghezzi, Genova (IT); Marco Dal Maschio, Genova (IT); Erica Lanzarini, Santa Sofia (Forli-Cesena) (IT); Guglielmo Lanzani, Milano (IT); Fabio Benfenati, Genova (IT)

(73) Assignee: Fondazione Istituto Italiano Di Tecnologia, Genova (IT)

(21) Appl. No.: 13/351,046

(22) Filed: Jan. 16, 2012

(51) Int. Cl. A61F 9/08; A61N 1/375

(52) U.S. Cl. 607/54; 607/62

(57) ABSTRACT

An interface for neuronal photoactivation includes a semiconducting polymer material, the semiconducting polymer material being excitable by luminous radiation for photovoltaically generating an electric signal. The semiconducting polymer material forms a substrate for neuronal cell adhesion.

### BACKGROUND OF THE INVENTION

The present invention relates to the field of neuroscience and biomedical technologies requiring the photostimulation of neurons and, more specifically, the field of artificial vision.

Investigated solutions for retinal diseases, caused by irreversible photoreceptors damage and leading to progressive loss of night vision, constricted visual fields, decreased visual acuity and ultimately to total blindness (like retinitis pigmentosa, RP, and age-related macular degeneration, MD), include gene therapy, transplantation and expression of optogenetic molecules. However, all these approaches obtained very partial results in clinical trials, and currently they cannot be used in medical therapies. There is currently no therapeutic cure for most forms of RP and MD, and implant of artificial prosthesis remains at the moment the sole, possible alternatives which have reached the development stage necessary for clinical trials.

However, all existing devices for neuronal photostimulation are based on inorganic technology, and they are affected by a number of major problems: the complexity of the fabrication processes, the limited flexibility, very poor biocompatibility, causing inflammation and gliosis reactions at the biological tissue level. Regarding in particular metal/silicon-based artificial prosthesis for sight restoration in blind patients, their success has been strongly limited by additional issues: electrodes have fixed, rigid geometries that do not conform to the natural curvature of the retinal tissue; implants can accommodate only a limited number of pixels, which severely limits the spatial resolution of the perceivable image; they have high impedance levels and generate high resistive currents, leading to rapid degradation, oxidation effects of the prosthesis itself and heat generation, very detrimental to the tissues, particularly in the case of the retinal one. Moreover, the contact between the artificial prosthesis and the natural tissue is very poor, they are scarcely tolerated, and they require in most cases an external power supply.

In this respect, organic soft matter has potential in terms of flexibility, favourable mechanical properties and biological affinity. The use of semiconducting polymers has been reported in mechanical actuators for precise delivery of neurotransmitters, and in biosensors, such as pH and glucose sensors, in which their ability to support mixed ionic/electronic charge transport was fully exploited. Conversely, organic polymers have been tested as coatings of conventional electrodes in direct neuronal interfaces for recording and stimulating neuronal activity, whereas the exploitation of their appealing optoelectronic features has been recently considered for neuronal communication and photo manipulation devices.

An interface device is proposed in the paper “A hybrid solid-liquid polymer photodiode for the bioenvironment” (M. R. Antognazza et al., Appl. Phys. Lett. 94, 243501 (2009)), wherein a semiconducting polymer material is used as a photodiode active material, said polymer material being poly[2-methoxy-5-(2′-ethylhexyloxy)-p-phenylene vinylene] (MEH-PPV) doped with phenyl-C61-butyric-acid-methyl ester (PCBM) and put in direct contact with electrolytic media of biological use, exploited for ionic charge transport, however without including a biological tissue.

### SUMMARY OF THE INVENTION

An object of the present invention is to provide an interface device for neuronal photoactivation, wherein functional interfacing between said organic device and neuronal cells is provided.

Accordingly, the invention proposes an interface for neuronal photoactivation comprising a semiconducting polymer material, said semiconducting polymer material being excitable by luminous radiation for photovoltaically generating an electric signal,
wherein said semiconducting polymer material forms a substrate for neuronal cell adhesion.

According to the invention, the inventors found that primary neurons can be successfully grown onto the polymer layer without affecting the optoelectronic properties of the active material or the biologic functionality of neuronal network. Moreover, action potentials can be triggered in a temporally reliable and spatially selective manner with short pulses of visible light.

A further object of the invention is a method for neuronal photoactivation, comprising the following steps:
providing neuronal cells arranged on a substrate,
irradiating the neuronal cells with a luminous radiation, and
transducing the luminous radiation into an electric signal for neuronal activation, wherein said transducing step is performed by a semiconducting polymer material, said semiconducting polymer material forming the substrate for adhesion of the neuronal cells.

A further object of the invention is a retinal prosthetic device for implantation on retina tissue, said device comprising a semiconducting polymer material which is excitable by luminous radiation for photovoltaically generating an electric signal,
wherein said semiconducting polymer material forms a substrate for neuronal cell adhesion.

The invention represents a fully original and revolutionary tool to overcome most of the limitations of conventional techniques and opens up a new way to realize efficient, organic-based retinal prosthetic devices.

In particular, the device according to the invention exploits the organic technology for the realization of an artificial photoreceptors layer. The photosensitive plastic device implantable either in the sub-retinic or in the epi-retinic space presents a number of advantages over the existing, silicon-based technology:
much higher biocompatibility and tolerability, thanks to materials flexibility, lightness and softness;
reduced invasivity and absence of rigid electrodes;
limited resistive currents and oxidation/reduction degradation processes at the electrodes;
low heat production;
no need for external power supply;
more intimate connection to living tissues thanks to the demonstrated phototransduction mechanism;
enhanced spatial resolution, with a continuous pattern of photostimulating sites;
potentially adjustable signaling timing;
possible coupling to existing flexible substrates, with already demonstrated biocompatibility and tolerability properties;
fabrication easiness.

Finally, with respect to existing optogenetic tools, the bio-organic interface does not require gene transfer, which is potentially hazardous and requires exact gene identification. For all these reasons, the invention represents a new tool for active, optically mediated neuronal interfacing, and can be exploited in any medical device requiring the photostimulation of a neuronal tissue, as an alternative to optogenetics techniques and prosthetic devices.

### BRIEF DESCRIPTION OF THE DRAWINGS

A preferred, but non-limiting, embodiment of the invention will now be described, with reference to the attached drawings, in which:

FIG. 1 show the operation principle of a solid-liquid organic photodetector according to the invention; in particular, FIG. 1a shows the chemical structures of regio-regular poly(3-hexylthiophene-2,5-diyl) (rr-P3HT) and phenyl-C61-butyric-acid-methyl ester (PCBM); FIG. 1b shows a scheme of hybrid solid-liquid cell, comprising a counter-electrode needed for modulated photocurrent spectroscopy experiments; FIG. 1c shows the spectral response of systems ITO/rr-P3HT:PCBM/NaCl/Gold (triangles) and ITO/rr-P3HT:PCBM/Ringer/Gold (squares), with the saline solutions working as ionic cathodes, recorded after 12 days of immersion in minimum essential medium. Typical spectral response (in arbitrary units for direct comparison with present results) of a conventional, solid-state solar cell (ITO/rr-P3HT:PCBM(1:1)/Al) is reported as a reference (dashed line); FIG. 1d shows photocurrent maxima (at 530 nm) recorded from eight distinct devices (represented in different colours by diamond symbols) immediately after fabrication (day 0) and up to 28 days later. Samples were kept completely immersed in the electrolyte for the whole period. Box plot limits represent the 25th and 75th percentile and whiskers represent the outliers (coefficient 1.5) of the distribution. The mean (square), maximum and minimum values of the distribution are also shown; FIG. 1e shows the stability of the photocurrent of the system ITO/rr-P3HT:PCBM/NaCl/Gold on continuous illumination. The data show the photocurrent recorded at 530 nm at various times up to 28 h;

FIG. 2 show cell viability on bioorganic active interface; in particular, FIG. 2a show hippocampal neurons cultured on either poly-L-lysine (PLL)-treated ITO/rr-P3HT:PCBMdevices (left) or control glass substrates covered with ITO and PLL only (right) after 12 days in vitro (DIV). Scale bars, 25 μm; FIG. 2b shows propidium iodine/fluorescein diacetate staining assay carried out at 21 DIV. Scale bars, 25 μm; FIG. 2c shows a statistical analysis of cell viability on control (left) or polymer-coated (right) substrates (P=0.558, one-way analysis of variance (ANOVA), n=6) at four time points: 7 DIV, 14 DIV, 21 DIV and 28 DIV; FIG. 2d shows a statistical analysis of cell mortality (P=0.445, one-way ANOVA, n=6) at the same time points of FIG. 1c;

FIG. 3 show the spontaneous electrical activity of primary hippocampal neurons cultured onto bioorganic active interfaces; in particular, FIG. 3a shows the resting membrane potential of neurons cultured on either poly-L-lysine (PLL)-treated ITO/rr-P3HT:PCBM devices (grey, n=24) or control glass substrates covered only with ITO and PLL (black, n=16) (t-test, P=0.506); FIG. 3b shows an analysis of the frequency (left; t-test, P=0.736, n=10) and amplitude distribution (right; n=6, bins 20 pA) of spontaneous excitatory postsynaptic currents (EPSCs); FIG. 3c shows an example of whole-cell recordings from neurons cultured on either polymer or control substrates. The analysis of neuronal firing rates in ITO/rr-P3HT:PCBM devices (n=10) and control substrates (n=10) did not show any significant difference (left, t-test, P=0.780); FIG. 3d shows the network mean firing rates (MFRs) computed with multielectrode array devices. Sample traces show a representative example of extracellular spikes recorded in the presence or absence of polymer deposition. Statistical analysis (t-test, P=0.843, n=9) did not show any difference between the two conditions. Scale bars, 30 μm. Box plot limits represent the 25th and 75th percentile and whiskers represent the outliers (coefficient 1.5) of the distribution. The mean (square), maximum and minimum values of the distribution are also shown; and

FIG. 4 show optical stimulation of neurons cultured onto an ITO/rr-P3HT:PCBM device; in particular, FIG. 4a shows a schematic representation of the optical stimulation paradigm including the localization of the stimulus in a region surrounding the patched neuron (scale bar, 10 μm); FIG. 4b shows a scheme of the photosensing interface, with the neuronal network grown on top of the polymer active layer during patch-clamp recordings; FIG. 4c shows the online monitoring of pH of the extracellular solution during the experiments in the presence (black, n=4) or absence of photostimulation (grey, n=4). Data are presented as means±s.e.m.; FIG. 4d shows action-potential generation in response to a photostimulation pulse (50 ms); FIG. 4e shows an example of spike train generated with 20 ms pulses repeated at 1 Hz (upper panel). Peristimulus time histogram (PSTH) count was computed and normalized by considering spike trains in all recorded neurons (bottom panel; n=10, bins 20 ms). The right plot shows (means±s.e.m.) the latency to the spike peak with respect to the light onset computed by averaging all spikes in the train obtained from all recorded neurons and the jitter calculated as the s.d. of spike latencies measured across all recorded neurons (n=10); FIG. 4f shows the spatial properties of the photostimulating interface. A grid of nine spots (diameter 20 μm, spacing 30 μm) was overlaid to a patched neuron and spikes were counted. PSTHs, arranged in a similar grid, represent the spike counts normalized for the total number of sweeps in all recorded neurons (n=10, bins 10 ms). Each histogram represents the count of the spikes recorded at the soma by the corresponding stimulation spot; FIG. 4g shows a model of the polymer/electrolyte and electrolyte/neuron interface, where Cᵢ and Rᵢ represent the capacitance and the resistance of the double layer at the interface of electrolyte and polymer, respectively. V_{b} represents the bias voltage traditionally applied for the cell stimulation (in our case V_{b}=0), Rₛ the electrolyte resistance and Vₘ the measured membrane potential. Grey bars and traces represent light pulses in all panels.

### DETAILED DESCRIPTION

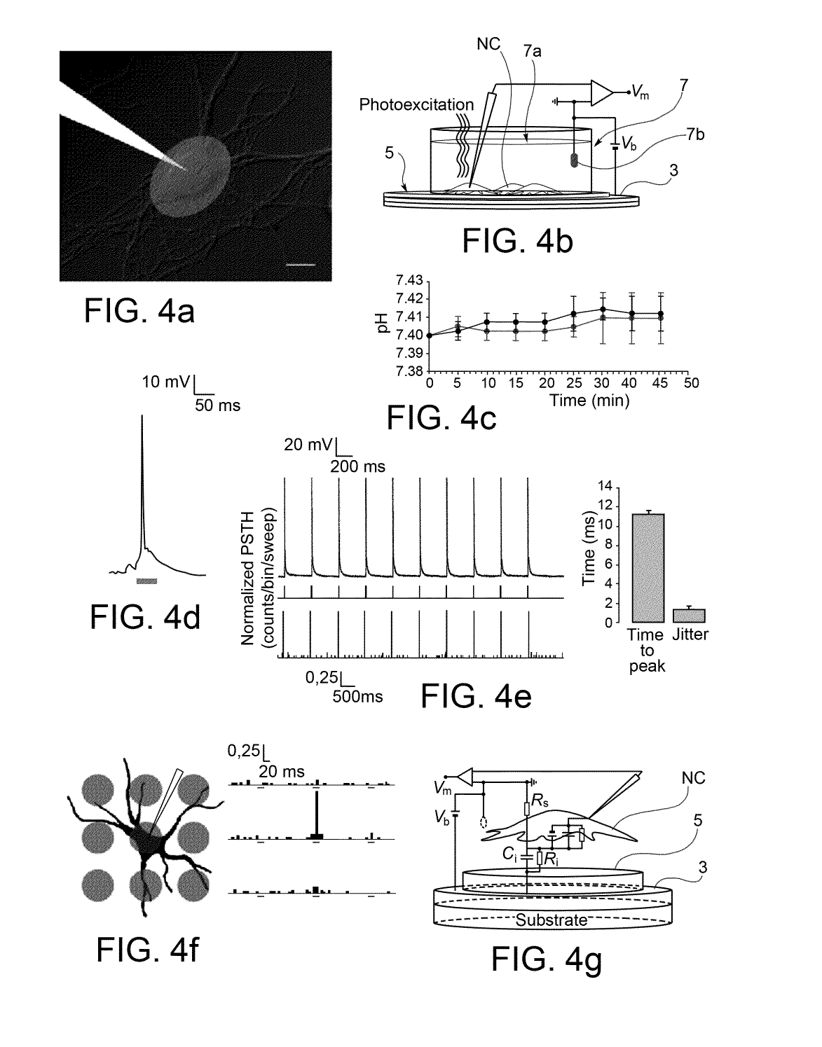

FIGS. 4b and 4g are schematic drawings representing a prototype of interface for neuronal photoactivation according to the invention. The interface shown in FIG. 4b comprises an anode layer 3 of transparent conducting material and a semiconducting polymer material 5 (active layer) coupled to the anode layer 3, said semiconducting polymer material being excitable by luminous radiation for photovoltaically generating an electric signal. Photocurrent measurements have shown that the anode layer could be omitted without appreciably affecting the operating principle of the device. The semiconducting polymer material and the optional anode layer may be arranged on a substrate of material which may be transparent to luminous radiation, depending on its operating configuration; in case the interface of the invention is conceived for a use as prosthetic retinal device, it is preferable that the material of the substrate, if any, is also a flexible one, for example a plastic material.

The prototype shown in FIG. 4b also comprises a cathode 7 comprising a liquid electrolyte 7a and, optionally, a counter electrode 7b for measuring neuronal activity. The liquid electrolyte 7a forms a culturing medium for neuronal cells, whereas the semiconducting polymer material 5 forms a substrate for neuronal cell adhesion. In FIG. 4b, neuronal cells grown on the polymer substrate are designated by NC. In case an interface according to the invention is conceived for a use as prosthetic retinal device, the cathode would be formed, in use, by the extracellular medium surrounding the neuronal cells.

Preferably, the semiconducting polymer material is regio-regular poly(3-hexylthiophene-2,5-diyl) doped with phenyl-C61-butyric-acid-methyl ester. Other suitable polymers include, for instance: poly(3-octylthiophene) (P3OT), poly[2-methoxy-5-(2′-ethylhexyloxy)-p-phenylene vinylene] (MEH-PPV), poly[2-methoxy-5-(3′,7′-dimethyloctyloxy)-1,4-phenylene vinylene] (MDMO-PPV), and other low-band gap polymers, e.g. for instance poly[2,6-(4,4-bis-(2-ethylhexyl)-4Hcyclopenta[2,1-b;3,4-b′]-dithiophene)-alt-4,7-(2,1,3-benzothiadiazole)] (PCPDTBT). Other possible electron acceptors include other fullerenes and fullerene derivatives, e.g. C60 and C70.

Preferably, the interface according to the invention further comprises an adhesion layer arranged on the semiconducting polymer material for improving adhesion of the neuronal cells. This adhesion layer may be composed, for example, of poly-L-Lysine.

As stated above, the structural and operating characteristics of the interface according to the invention may be applied in the realization of a retinal prosthetic device for implantation on retina tissue, wherein the semiconducting polymer material forms a substrate for neuronal cell adhesion.

This photosensitive, plastic prosthetic device would be in principle implantable either in the sub-retinic or in the epi-retinic space, and present a number of advantages over the existing, silicon-based technology:
much higher biocompatibility and tolerability, thanks to materials flexibility, lightness and softness;
reduced invasivity and absence of rigid electrodes;
limited resistive currents and oxidation/reduction degradation processes at the electrodes;
low heat production;
no need for external power supply;
more intimate connection to living tissues thanks to the demonstrated phototransduction mechanism;
enhanced spatial resolution, with a continuous pattern of photostimulating sites;
potentially adjustable signaling timing;
possible coupling to existing flexible substrates, with already demonstrated biocompatibility and tolerability properties;
fabrication easiness.

A prototype device according to the invention has been realized by the inventors. In the following sections, the realization process of such a prototype, as well as tests and measurements made therewith, are disclosed.

Realization of the Bioorganic Interface

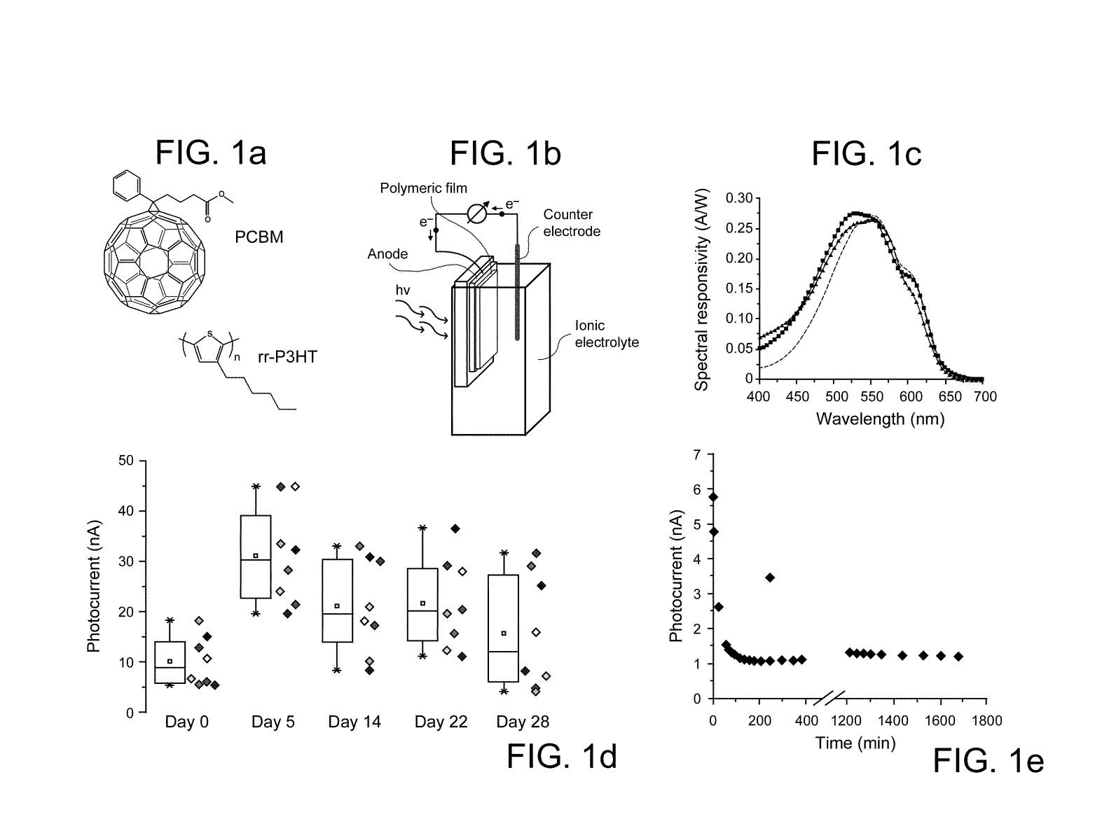

As active layer of the interface regio-regular poly(3-hexylthiophene-2,5-diyl) doped with phenyl-C61-butyric-acid-methyl ester (rr-P3HT:PCBM) (FIG. 1a) was used. In the bulk heterojunction structure, rr-P3HT works as an electron donor material, whereas PCBM is the electron acceptor, thus ensuring very high external quantum efficiency in the charge generation process.

The device was realized through a multi-stage process: the active polymer film was spin-coated onto a glass substrate pre-coated with indium-tin oxide (ITO), which works as the anode of the photo-detector, and then the organic blend was annealed at 120° C. for 2 h. The thermal treatment had a double role: it improved the morphology of the polymeric film, enhancing the efficiency of charge photogeneration, and it prepared the film for subsequent cell culture by removing all residual traces of organic solvents (for example, acetone, methyl alcohol, chlorobenzene, which are highly toxic for the biological systems) and by sterilizing the substrate. The polymer layer was covered by poly-L-lysine (PLL) to improve adhesion.

Both rr-P3HT and PCBM were supplied by Sigma-Aldrich and used without any further purification. rr-P3HT has a regio-regularity of 99.5% and molecular weight of 17,500 g mol⁻¹. An accurate cleaning of the substrate was required: the substrate was rinsed in an ultrasonic bath with, subsequently, a specific tension-active agent in water solution (HELLMANEX II, 3%), deionized water, pure acetone and isopropyl alcohol. Oxygen plasma cleaning of the substrate completed the process. 1,2-Chlorobenzene solutions of P3HT and PCBM were prepared separately, with a concentration of 7.5 g l⁻¹, and then mixed together (1:1 volume ratio) using a magnetic stirrer. The solution was then heated at 50° C., stirred and finally deposited on the ITO-covered (thickness 0.4 mm) glass substrate, previously heated, by spin coating. Spinning parameters (first step: 800 r.p.m., angular acceleration 1,500 rad s⁻², rotation duration 2 s; second step: 1,500 r.p.m., angular acceleration 4,000 rad s⁻², rotation duration 30 s) were carefully selected to obtain suitable optical quality and film thickness (˜150 nm). Polymers on ThinMEA200/30 (Multi Channel Systems MCS GmbH) were deposited in the same way. After deposition, organic layers were annealed and properly sterilized by heating at 120° C. for 2 h. Control substrates (ITO-covered glass substrates) were properly sterilized in the same way.

Primary rat embryonic hippocampal neurons were finally seeded and grown on the polymer layer of PLL. Primary cultures of hippocampal neurons were prepared from embryonic 18-day rat embryos (Charles River). Briefly, hippocampi were dissociated by a 15-min incubation with 0.25% trypsin at 37° C., and cells were plated at a density of 275-300 cells per mm² on PLL (0.1 mg ml-1)-treated organic layers or control substrates in Neurobasal (Invitrogen) supplemented with 10% Horse serum (Hyclone). ThinMEA200/30 (Multi Channel Systems MCS GmbH) were treated with PLL and neurons were plated at a density of 400-500 cells per mm². After allowing neurons to adhere to the surface for 3-4 h, cells were cultured in serum-free Neurobasal supplemented with 2% B27 (Invitrogen), 2 mM Glutamine (Invitrogen) and 1% Penicillin/streptomycin mixture (Invitrogen).

Assessment of the Organic Semiconductor Functionalities

Specific requirements have to be fulfilled by a successful device based on organic semiconductors: the organic semiconductor must withstand sterilization procedure; the organic semiconductor must preserve its characteristics once immersed in culture medium; cell growth and survival must be demonstrated on top of the organic active layer; and proper functionalities of the semiconducting layer and of the neuronal network have to be demonstrated.

To address the first two issues, the inventors investigated prototypes without neurons, which had undergone the same fabrication steps. The inventors used hybrid solid-liquid devices (FIG. 1b) maintained before the experiment in minimum essential medium (a standard and widely used culture medium) and using an aqueous solution of sodium chloride (NaCl) as the electrolytic cathode. Given that NaCl represents the main ionic content of any extracellular fluid, the latter solution was used as a simpler and proper model system for understanding the hybrid photodetector operation principle. FIG. 1c demonstrates that the photosensing capabilities of the organic polymer are fully preserved after thermal sterilization, casting of PLL and direct contact with saline solutions for 12 days. FIGS. 1d and 1e show the temporal stability and photostability of the hybrid cell, respectively. The inventors verified that the active polymer blend maintains its photoconversion capability unaltered for 28 days in response to light, while remaining completely immersed in the electrolytic solution for the whole period (FIG. 1d). Moreover, the inventors also verified the effect of a continuous illumination for more than 28 h on the photoconversion efficiency and found that, after a transient decay at the very onset of the illumination, the photocurrent values were very stable over time (FIG. 1e).

In the modulated photocurrent spectroscopy, a 30-W halogen tungsten lamp served as a source of white light, filtered through a monochromator before being focused on the polymer film. The light was chopped by a mechanical chopper, whose reference signal was fed to a lock-in amplifier with detection at the chop frequency (270 Hz). All measurements were taken at room temperature without applying bias. The hybrid device (FIG. 1b) had a vertical geometry with an ITO layer that served as an anode, the active polymer deposited on top of it, and an aqueous NaCl (200 mM) solution used as the electrolytic liquid cathode. Other solutions tested for the cathode included: minimum essential medium, recording extracellular solution (Ringer, see below) and several aqueous solutions with various concentrations of other salts like NaI or NaBr. The photocurrent was extracted and measured in the external circuit through a counter-electrode (gold) immersed in the electrolyte.

Polymer Effect on Cell Viability

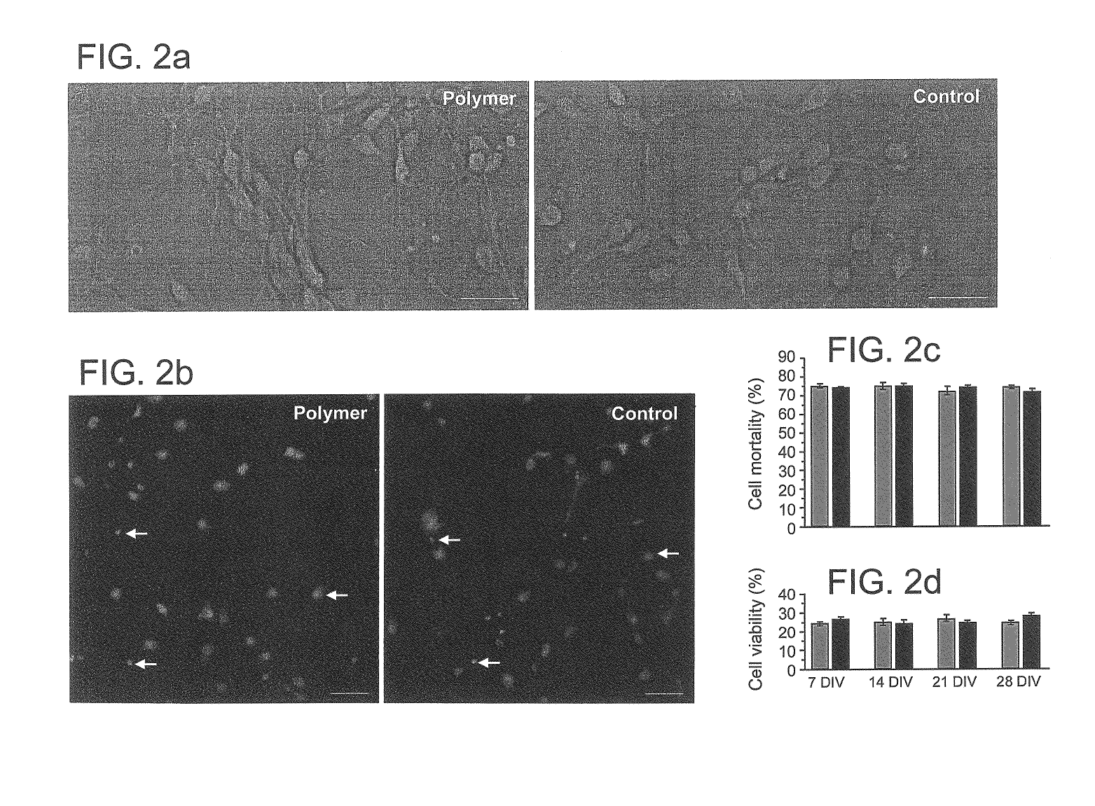

To address the issue of biocompatibility of the organic layer, primary neurons were grown for up to 28 DIV onto either PLL-treated ITO/rr-P3HT:PCBM devices (polymer) or control glass substrates covered with ITO and PLL only (control; FIG. 2a). Neuronal adhesion to the substrate and growing pattern on ITO/rr-P3HT:PCBM devices were virtually identical under both conditions. To compare the cell viability on ITO/rr-P3HT:PCBM devices with respect to control substrates, propidium iodine/fluorescein diacetate staining was carried out at various times in culture (FIG. 2b). No significant differences were found in both cell viability (FIG. 2c, P=0.558, one-way ANOVA, n=6) and mortality under both culture conditions (FIG. 2d, P=0.445, one-way ANOVA, n=6), indicating that the organic polymer alters neither the network development nor the neuronal viability over a long period of time.

Cell viability was verified by propidium iodine/fluorescein diacetate staining at 7 DIV, 14 DIV, 21 DIV and 28 DIV. Attached cells were rinsed twice with Ringer and incubated for 4 min in Ringer containing 15 μg ml-1 of Fluorescein diacetate (Sigma-Aldrich), 5 μg ml-1 of Propidium iodine (Sigma-Aldrich) and 3 μg ml-1 of Hoechst-33342 (Sigma-Aldrich). Cells were washed with Ringer and multiple images were taken by a C91006 CCD Camera (Hamamatsu Photonics Italia) mounted on a Nikon Ti-E epifluorescence microscope (Nikon Instruments). Standard 4′-6-diamidino-2-phenylindole (ex: D350/50x, em: D460/50m, dic: 400dclp), fluorescein isothiocyanate (ex: D480/30x, em: D535/40m, dic: 505dclp) and tetramethylrhodamine isothiocyanate (ex: D540/25x, em: D605/55m, dic: 565dclp) filter sets were used to image Hoechst-33342, fluorescein diacetate and propidium iodine, respectively.

At each time point, images were taken from both organic layers (n=6) and control substrates (n=6). Five fields per substrate were imaged and the percentage of living (or dead) cells per field was counted and averaged. The percentage of healthy cells was calculated as the percentage of cells expressing fluorescein diacetate on the total nuclei expressing Hoechst-33342. As a control, the percentage of dead cells was computed as the percentage of apoptotic cells (identified by Hoechst-33342) and necrotic cells (identified by propidium iodine) on the total number of nuclei expressing Hoechst-33342. Percentage of living cells (or dead cells) was reported at every time point as mean±s.e.m. of six independent plates.

Polymer Effect on Neuronal Physiology

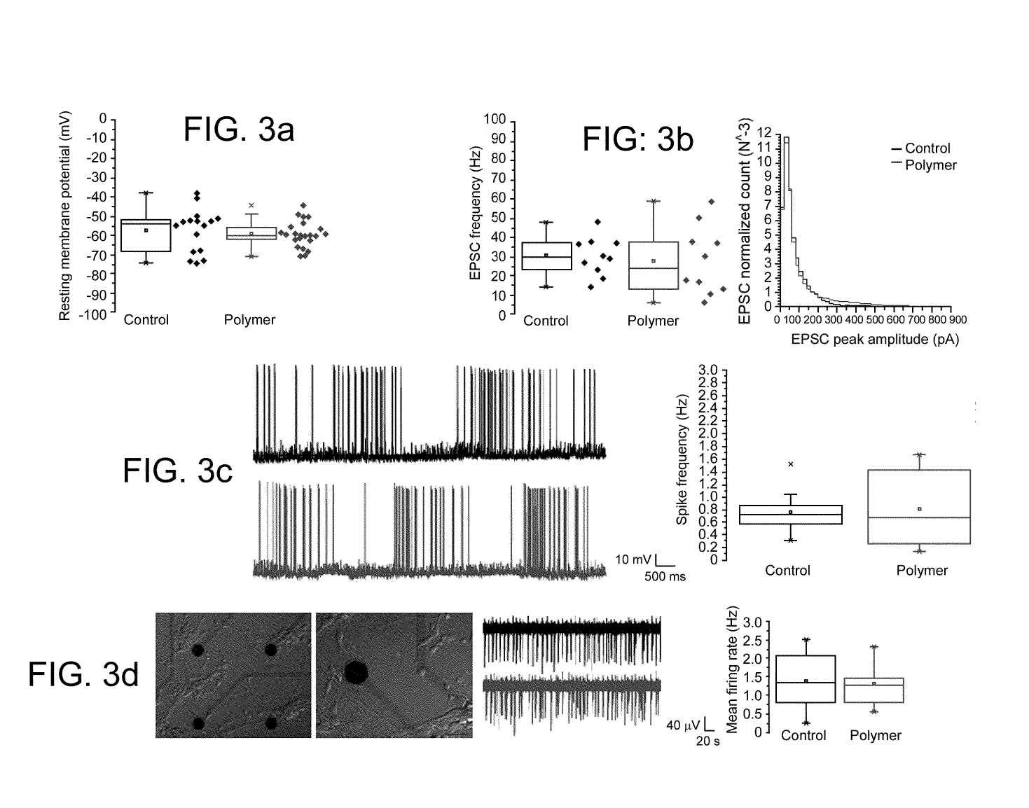

The physiological properties of neurons cultured on bioorganic active interfaces were evaluated by using the standard patch-clamp technique. Both the resting membrane potential (FIG. 3a) and the frequency and amplitude distribution of spontaneous excitatory postsynaptic currents (FIG. 3b) were closely similar under the two culture conditions. Moreover, the firing rate measured in current-clamp mode (FIG. 3c) did not reveal any significant difference between the two conditions. To characterize network behaviour under the two conditions, neurons were plated onto microelectrode array biochips and the network mean firing rate was measured after 21 DIV (FIG. 3d). No differences in spontaneous network firing were observed between uncoated and polymer-coated multielectrode arrays (t-test, P=0.843, n=9). These results demonstrate that organic layers are highly biocompatible and fully preserve the fundamental physiological characteristics of mature neurons.

Electrophysiology was performed on a setup based on a Nikon FN1 upright microscope (Nikon Instruments). Differential interference contrast (DIC) images were taken by a C91006 CCD Camera (Hamamatsu Photonics Italia). Whole-cell patch-clamp recordings were performed between 14 DIV and 18 DIV at room temperature using patch pipettes (4-6 MΩ), under GΩ patch sealing, using an Axopatch 200B (Axon Instruments). The recording extracellular solution (Ringer) contained (mM): NaCl 135, KCl 5.4, MgCl2 1, CaCl2 1.8, HEPES 5, glucose 10 and pH was adjusted to 7.4 with NaOH. The intracellular solution contained (mM): KCl 140, HEPES 5, EGTA 5, MgCl2 2 and pH was adjusted to 7.35 with KOH. Responses were amplified, digitized at 20 kHz and stored with pCLAMP 10 (Axon Instruments). Further analyses were carried out using pCLAMP 10 and Matlab (The MathWorks).

Excitatory postsynaptic currents were recorded in voltage-clamp mode by applying a—70-mV holding. Peaks were detected in 5 min recordings per cell by a threshold algorithm implemented in Matlab. ESPCs distribution was normalized by the area. Single-cell spike frequency was evaluated by 5-min current-clamp recording per cell.

Microelectrode array recordings were performed in the culturing medium at 37° C. using the MEA1060-inv-BC system (Multi Channel Systems MCS GmbH). Data recorded at 25 kHz ch—1 from the 60 channels were filtered from 200 Hz to 3 kHz and spikes were sorted using a threshold algorithm included in the MC Rack software (Multi Channel Systems MCS GmbH). The threshold per every electrode was defined as a multiple of the s.d. of the biological noise computed during the first 500 ms of the recording (−5×s.d.ₙₒᵢₛₑ). Global mean firing rate of the network in a multielectrode array chip was computed as mean of the firing rates obtained for every active channel present in the chip. pH measurements were taken during electrophysiological experiments, in both organic layers and controls substrates, using a PCE-228 (PCE Italia) pH metre equipped with a BEE-CAL compensator (World Precision Instruments) and a Beetrode wire pH electrode (World Precision Instruments). Data are always presented as mean±s.e.m.

Statistical analysis was carried out using SigmaPlot (Systat Software), and data were plotted using OriginPro (OriginLab Corporation) softwares. Data were compared by Student's t-test or one-way ANOVA.

Neuronal Photostimulation

The inventors tested the performance of the interface structure as an intercommunication device by evaluating the efficacy of light excitation to trigger the activity of whole-cell patched neurons (FIG. 4a). Whole-cell recordings were carried out (FIG. 4b) under photostimulation in photovoltaic mode (Vb=0 V). First, the inventors checked that the photostimulation mechanism did not affect the cellular microenvironment. When the extracellular pH was monitored in close proximity to the recorded cells in the presence or absence of photostimuli, the local pH did not significantly drift over 0.02 pH units (FIG. 4c), a change which is unlikely to affect neuronal function. Moreover, a deterministic correspondence between photostimulation of the organic semiconductor close to the cell body and neuronal activation was found (FIG. 4d). Spike trains (FIG. 4e, upper panel) could be elicited with trains of light pulses (20 ms at 1 Hz) with a negligible percentage of failures (<15% on the total number of delivered pulses; see FIG. 4e bottom panel). Single spikes were rapidly and precisely evoked (time to peak: 11.324±0.171 ms; jitter: 1.481±0.258 ms). The spatial selectivity of photostimulation was investigated by aligning a 3×3 stimulation grid to the neuronal cell body of each optically stimulated neuron (n=10) under whole-cell recording (FIG. 4f, left). Peristimulus time histograms show that action potentials were reliably evoked only when the stimulus spot was precisely hitting the cell body, whereas the stimulus was virtually ineffective when the spot was translated by a cell body diameter in all directions (FIG. 4f, right).

Using a custom LabView application (National Instruments), a specific region of interest surrounding the soma of a selected neuron was drawn and the resulting binary image was used to control a Digital Micromirror Device (DMD, Texas Instruments). The DMD was in turn used to shape a Laser beam (Cobolt) resulting in a selective photostimulation limited to the defined region of interest. Laser pulses (532 nm, 10 mW mm⁻²) were timed using a Mechanical shutter (Uniblitz) and delivered to the sample through a 40×/0.8NA water immersion objective (Nikon Instruments).

Discussion

The physical mechanism underlying the photostimulation process (FIG. 4g) could be either a pure resistive (Rᵢ) behaviour or a pure capacitive (Cᵢ) behaviour, or a combination of the two. In the first scenario, following polymer photoexcitation and photocurrent generation, the charge migration from the polymer to the electrolyte gives rise to Faradaic currents; subsequently, the ionic unbalance created in the extracellular medium can give rise to membrane depolarization and subsequent firing. This scenario is usually taken into consideration whenever an external bias, stronger than the relevant electrochemical potentials and high enough to enable water hydrolysis (in our case higher than 3.4 V), is applied. Note that Faradaic processes resulting from reduction—oxidation electrochemical reaction between species in solution and electrodes might induce both electrode damage and cell degradation, and for this reason these processes should be avoided. The photostimulation configuration relying on a pure capacitive mechanism is characterized by the generation of two oppositely charged Helmholtz layers at the electrolyte/polymer and neuronal membrane/electrolyte interfaces, without charge transfer processes between the polymer and the electrolyte. A charge displacement within the polymeric bulk induces ion redistribution at the electrolytic/cell membrane interface, thus leading to membrane depolarization with subsequent action-potential generation.

Considering that the device operates in photovoltaic mode, the recorded photocurrent is low (in the order of hundreds of pA) and as the inventors did not observe any adverse effect during the stimulation experiments (neither modification of the polymeric film, nor of the neuronal culture), the capacitive coupling is the most likely mechanism of stimulation. Although a Faradaic component of the current cannot be completely excluded, in the present case it seems to be negligible; if present, a significant increase in the concentration of hydroxide ions is expected, in contrast to the experimental evidence.

In conclusion, a new communication protocol between organic semiconductors and neuronal cells showing photostimulation of neuronal activity is demonstrated. The present approach has advantages with respect to similar attempts based on biased devices. In contrast to metal or silicon interfaces, the proposed interface works without any externally applied electric field and with minimal heat dissipation, favourably addressing the thermal issues, which are extremely relevant in an efficient biological interface.

According to the present invention, r-conjugated materials can be taken into consideration for a new generation of biomimetic artificial retinal prostheses. Organic technology is characterized by simple and cheap fabrication techniques; existing deposition methods such as ink jet printing, allow the realization of a variety of geometrical patterns with various active areas, up to few square micrometres, thus offering the possibility to specifically target selected groups of cells.

The use of soft matter provides some advantages in terms of mechanical properties, as it enables the production of light, thin and flexible devices, better suited to implantation within a biological environment. Moreover, in terms of photoconversion efficiency, the appropriate stimulatory light to induce polymeric photostimulation can be obtained and modulated by using a combination of camera detectors and light emitters as enhancers (MicroLEDs), thus ensuring that the retinal prosthetic device can work in a wide-band range of ambient light intensities.

The present approach represents a new tool for neuronal active interfacing, which is a simpler alternative to the existing and widely used neuron optogenetic photostimulation techniques, and avoids gene transfer, which is potentially hazardous. The photostimulation is not specific for selected neuronal populations, as is this case for genetically encoded approaches, but the optical stimulation of neurons could be micrometrically shaped to stimulate selected neuronal populations owing to the high spatial selectivity of the photostimulation interface, and could lead to the development of new artificial optoelectronic neurointerfaces based on biocompatible organic materials.

What is claimed is:

1. An interface for neuronal photoactivation comprising a semiconducting polymer material, said semiconducting polymer material being excitable by luminous radiation for photovoltaically generating an electric signal,
wherein said semiconducting polymer material forms a substrate for neuronal cell adhesion.

2. An interface according to claim 1, further comprising an anode layer of transparent conducting material coupled to said semiconducting polymer material.

3. An interface according to claim 1, further comprising a cathode comprising a liquid electrolyte.

4. An interface according to claim 1, wherein said semiconducting polymer material comprises a polymer selected from the group consisting of regio-regular poly(3-hexylthiophene-2,5-diyl) (rr-P3HT), poly(3-octylthiophene) (P3OT), poly[2-methoxy-5-(2′-ethylhexyloxy)-p-phenylene vinylene] (MEH-PPV), poly[2-methoxy-5-(3′,7′-dimethyloctyloxy)-1,4-phenylene vinylene] (MDMO-PPV), poly[2,6-(4,4-bis-(2-ethylhexyl)-4Hcyclopenta[2,1-b; 3,4-b′]-dithiophene)-alt-4,7-(2,1,3-benzothiadiazole)] (PCPDTBT).

5. An interface according to claim 4, wherein said polymer is doped with an electron acceptor material selected from the group consisting of fullerenes and fullerene derivatives.

6. An interface according to claim 1, further comprising an adhesion layer arranged on the semiconducting polymer material for improving adhesion of neuronal cells.

7. A retinal prosthetic device for implantation on retina tissue, said device comprising a semiconducting polymer material which is excitable by luminous radiation for photovoltaically generating an electric signal,
wherein said semiconducting polymer material forms a substrate for neuronal cell adhesion.

8. A device according to claim 7, further including an anode layer of transparent conducting material coupled to said semiconducting polymer material.

9. A device according to claim 7, further including, in use, a cathode formed by an extracellular medium.

10. A device according to claim 7, wherein said semiconducting polymer material comprises a polymer selected from the group consisting of regio-regular poly(3-hexylthiophene-2,5-diyl) (rr-P3HT), poly(3-octylthiophene) (P3OT), poly[2-methoxy-5-(2′-ethylhexyloxy)-p-phenylene vinylene] (MEH-PPV), poly[2-methoxy-5-(3′,7′-dimethyloctyloxy)-1,4-phenylene vinylene] (MDMO-PPV), poly[2,6-(4,4-bis-(2-ethylhexyl)-4Hcyclopenta[2,1-b;3,4-b′]-dithiophene)-alt-4,7-(2,1,3-benzothiadiazole)] (PCPDTBT).

11. A device according to claim 10, wherein said polymer is doped with an electron acceptor material selected from the group consisting of fullerenes and fullerene derivatives.

12. A device according to claim 7, further comprising an adhesion layer arranged on the semiconducting polymer material for improving adhesion of neural cells.

13. A method for neuronal photoactivation, comprising the following steps:
providing neuronal cells arranged on a substrate,
irradiating the neuronal cells with a luminous radiation,
transducing the luminous radiation into an electrical signal for neuronal activation,
wherein said transducing step is performed by a semiconducting polymer material, said semiconducting polymer material forming the substrate for adhesion of the neuronal cells.

14. A method according to claim 13, wherein said semiconducting polymer material comprises an electron donor material selected from the group consisting of regio-regular poly(3-hexylthiophene-2,5-diyl) (rr-P3HT), poly(3-octylthiophene) (P3OT), poly[2-methoxy-5-(2′-ethylhexyloxy)-p-phenylene vinylene] (MEH-PPV), poly[2-methoxy-5-(3′,7′-dimethyloctyloxy)-1,4-phenylene vinylene] (MDMO-PPV), poly[2,6-(4,4-bis-(2-ethylhexyl)-4Hcyclopenta[2,1-b;3,4-b′]-dithiophene)-alt-4,7-(2,1,3-benzothiadiazole)] (PCPDTBT).

15. A method according to claim 14, wherein said polymer is doped with an electron acceptor material selected from the group consisting of fullerenes and fullerene derivatives.

16. A method according to claim 13, wherein said interface further comprises an adhesion layer arranged on the semiconducting polymer material for improving adhesion of neuronal cells.
